(19) United States
(12) Patent Application Publication
(10) Pub. No.: US 2026/0103674 A1
(43) Pub. Date: Apr. 16, 2026

(54) A METHOD OF CULTURING LAWSONIA INTRACELLULARIS BACTERIA

(71) Applicant: INTERVET INC., Rahway, NJ (US)

(72) Inventors: Ad DE GROOF, Boxmeer (NL); Bartjan Willem SIMMELINK, Boxmeer (NL); Jelle STERK, Boxmeer (NL)

(73) Assignee: INTERVET INC., Rahway, NJ (US)

(21) Appl. No.: 19/115,281

(22) PCT Filed: Sep. 26, 2023
(86) PCT No.: PCT/EP2023/076554

§ 371 Date: Mar. 26, 2025

(30) Foreign Application Priority Data
Sep. 27, 2022 (EP) 22197915.6

(51) Int. Cl. C12N 1/205; C12R 1/01

(52) U.S. Cl. CPC C12N 1/205; C12R 2001/01

(57) ABSTRACT

The present invention relates to a method of culturing Lawsonia intracellularis. To obtain a more stable and constant production yield, McCoy cells are infected with Lawsonia intracellularis bacteria, grown in a first growth medium at a dissolved oxygen concentration less than 18% to arrive at a first culture of McCoy cells infected with Lawsonia intracellularis bacteria. The concentration of pyroptotic McCoy cells in the first culture is determined, and a first portion of the first culture is passed to a second growth medium containing fresh McCoy cells, such that the second growth medium is inoculated with 0.05-0.25 pyroptotic McCoy cells per fresh McCoy cell. The McCoy cells in the second growth medium are grown at a dissolved oxygen concentration less than 18% to arrive at a second culture of McCoy cells infected with Lawsonia intracellularis bacteria.

### FIELD OF THE INVENTION

The present invention relates to a method of culturing Lawsonia intracellularis.

### BACKGROUND

Lawsonia intracellularis (also called Lawsonia) is the causative agent of proliferative enteropathy (also called enteritis or ileitis) in many animals, in particular pigs, and presents a clinical sign and pathological syndrome with mucosal hyperplasia of immature crypt epithelial cells, primarily in the terminal ileum. Other sites of the intestines that can be affected include the jejunum, caecum and colon. Weanlings and young adult pigs are principally affected with typical clinical manifestation of rapid weight loss and dehydration. Natural clinical disease in pigs occurs worldwide. The disease is consistently associated with the presence of intracellular curved bacteria, presently known as Lawsonia. Disinfectants are used to inactivate Lawsonia bacteria, while antibiotics are used for prophylaxis or treatment of ileitis. Furthermore, nutritional supplements are used to promote a beneficial gut microbiome. In general, however, vaccination against Lawsonia has shown to be an economically efficient measure to treat an infection with Lawsonia. This way ileitis can be controlled, allowing a better exploitation of the genetic growth potential of the pig.

It would therefore be desirable to have an efficient and economically attractive cultivation method for these intracellular bacteria for commercial production of substantive volumes of a vaccine to treat an infection with Lawsonia.

EP 843 818 describes a method to grow Lawsonia bacteria in McCoy cells adhered to a substrate (such as a flask bottom or micro-carriers). Indeed, McCoy cells have proven to be adequate host cells to cultivate Lawsonia bacteria. It is also known from this patent that for passage of a culture to actually grow the Lawsonia bacteria (i.e. to increase the net number of viable bacteria), one needs to incubate fresh uninfected McCoy cells at low oxygen, that is below 18%, whereafter these fresh cells are inoculated with already infected McCoy cells. Passage to fresh McCoy cells is believed to be necessary since the infected McCoy host cells are ultimately killed by the intracellular Lawsonia bacteria. Indeed, it has been described that a Lawsonia infection spreads via host cells that lyse and therewith release the intracellular bacteria such that they become available for infection of other cells.

EP 843 818 also describes to select the volume of infected culture to be used as an inoculum for fresh McCoy cells depending on the concentration of Lawsonia bacteria present in the medium of the culture of infected McCoy cells.

A disadvantage of this method is however that the yield of the cultivation process varies substantially, even if the passaging is consistently based on the same ratio of Lawsonia bacteria and fresh McCoy cells (multiplicity of infection, MOI). This is inconvenient, since a reliable and stable supply yield is required for vaccine production and complying with vaccine demand.

### DESCRIPTION OF THE INVENTION

Thus, there is still a need for an improved cultivation technique for Lawsonia intracellularis bacteria.

To this end, a method of culturing Lawsonia intracellularis bacteria is provided, wherein Lawsonia intracellularis bacteria infect McCoy cells, and the infected McCoy cells are grown in a first growth medium and at a dissolved oxygen concentration of less than 18% to arrive at a first culture of McCoy cells infected with Lawsonia intracellularis bacteria. Then the number of pyroptotic McCoy cells per volume unit of the first culture is determined (either directly by measuring actual pyroptosis of the cells or indirectly by measuring a parameter that has a known relationship with pyroptosis for the McCoy cells). A second growth medium containing fresh McCoy cells is provided, and the volume of first culture is determined in order to provide the fresh McCoy cells with pyroptotic cells in a ratio of 0.05-0.25 pyroptotic McCoy cells per fresh McCoy cell. A first portion of the first culture comprising the required amount of pyroptotic cells is transferred to the second growth medium, for arriving at a second culture of McCoy cells infected with Lawsonia intracellularis bacteria. The second culture is grown at a dissolved oxygen concentration of less than 18%.

Surprisingly, it was found that it was not the number of Lawsonia bacteria present in the medium of the culture of McCoy cells infected with Lawsonia intracellularis bacteria which is decisive for a more stable and constant production yield of the cultivation process, but rather the physiological state of the McCoy cells of the culture of McCoy cells infected with Lawsonia intracellularis bacteria at the moment of passing a first portion of the first culture of McCoy cells to growth medium containing fresh McCoy cells, in particular whether or not the McCoy host cells are pyroptotic.

Pyroptosis is a form of programmed cell death that is triggered by proinflammatory signals and involves activation of caspase-1. It was found that when the second growth medium containing fresh McCoy cells was inoculated with in a ratio of pyroptotic McCoy cell:fresh McCoy cell of (0.05:1)-(0.25:1), the fresh McCoy cells were effectively infected with Lawsonia bacteria, thus continuing the culture process, while the remainder of the first culture of McCoy cells contained a relatively high number of Lawsonia bacteria for harvesting for vaccine production. Notably, this resulted in both a higher average yield, and a reduced variability in yield between cultures, and therefore also reduced production costs of e.g. vaccines requiring the cultivation of Lawsonia intracellularis bacteria.

The optimum ratio of pyroptotic McCoy cells and fresh McCoy cells depends i.a. on the number of days the second culture is to be grown, for example whether the second culture is to be used for passing a first portion of the first culture to fresh medium containing fresh McCoy cells on a predetermined day, or if the second culture is to be used for harvesting Lawsonia bacteria on that or another day. The ratio of pyroptotic McCoy cells to fresh McCoy cells may for example be 0.05:1, 0.06:1, 0.07:1, 0.075:1, 0.08:1, 0.09:1, 0.1:1, 0.125:1, 0.15:1, 0.2:1, 0.225:1 or 0.25:1, in order to adapt to a specific combination of number of fresh McCoy cells, volume of the fresh medium and number of days the second culture is planned to be grown. Those variabilities fall in the routine ambit of any person skilled in the art of cell culture.

Depending on the concentration of pyroptotic McCoy cells in the first culture, the concentration of fresh McCoy cells in the second growth medium and the number of days the second culture is to be grown, the first portion of first culture that is passed to the second growth medium may be any amount ranging from 1%-100% of the volume of the first culture, such as 10%, 20%, 25%, 30%, 35%, 40%, 45%, 50%, 60%, 70%, 75%, 80%, 85%, 90%, or 95%.

It is noted that many media are known that support the growth of McCoy cells as well as Lawsonia bacteria. It is commonly known how to constitute a medium that supports growth of cells or bacteria. For cells, classical culture media were originally developed by Eagle, Ham and others in the 1950's and 60's. They found that a medium which fulfils the basic needs for growth should comprise inorganic salts, a nitrogen source (for example in the form of nitrogen containing compounds such as peptides or proteins), a carbon source and vitamins. The media are advantageously buffered to prevent them from becoming either too acidic or too alkaline. Within this basic recipe, many different constitutions are available. For example, one could opt for animal derived components to provide the amino acids, but one could also choose for chemically defined amino acids. For the other compounds numerous variations are possible as well. To cultivate bacteria even less requirements are necessary. In short, to constitute a medium that supports growth of cells or bacteria is relatively simple.

However, optimization of growth and/or metabolite production can take some time, in particular when a medium is preferred that is free of serum or other animal derived components. However, the type of work is routine: strategies for improving medium performance however are commonly known in the art and elaborately described in literature (see for example a review article by Kennedy and Krouse in the Journal of Industrial Microbiology & Biotechnology (1999) 23, 456-475). A medium as simple as the commonly known DMEM may be sufficient to apply the present invention, optionally supplemented with e.g. glucose, one or multiple growth factors or essential amino acids such as L-glutamine or cysteine, and/or antibiotics.

The environment is also not very critical, although a dissolved oxygen concentration below 18% (that is below the dissolved oxygen concentration in balance with normal atmospheric oxygen) is necessary to obtain adequate growth of Lawsonia intracellularis bacteria. Preferably the dissolved oxygen concentration is as low as 0, 1, 2, 2.5, 3, 4, 5, 6, 7, 7.5, 8, 9, 10, 11, 12, 12.5, 13, 14, 15, 16, 17, or 17.5%.

It is also noted that passing culture cells (also referred to as passaging) is a completely different physiological process than merely adding fresh medium to cells in an existing culture. Adding fresh medium to an existing culture (for example such as known from WO 2006/056853, example 1) is done merely to provide nutrients to enable the existing cells to grow and stay alive. Passing means that cells are transferred to fresh culture medium in a new culture vessel, typically containing fresh (uninfected) culture cells.

### Definitions

McCoy cell: a fibroblast of mouse origin (e.g. ATCC CRL-1696).

Fresh McCoy cells: McCoy cells that are not infected by Lawsonia intracellularis bacteria.

To culture: to maintain a population of cells.

To pass: to re-seed cultured cells into fresh medium, typically containing fresh culture cells, in order to support growth of the cells.

To grow: to net increase in the amount of viable cells.

To harvest: to collect cells for further processing to arrive at an end product containing the harvested (processed) cells.

Growth medium: medium suitable to maintain viable cells and to support growth of the cells.

Suspension: a state wherein cells are present in a medium without being adhered to a surface.

Pyroptosis: inflammatory form of programmed cell death involving activation of caspase-1.

Pyroptotic cell: cell in the process of undergoing pyroptosis.

Viable cell: a cell capable of growing and dividing.

Viability: ratio of viable cells over the sum of viable and not-viable cells.

Viable cell density: number of viable cells per unit of volume.

### Embodiments of the Invention

In an embodiment, the McCoy are grown in suspension.

By maintaining the McCoy cells in a suspended state during incubation, maximum growth of the McCoy cells, and thus Lawsonia, is achieved by increasing each individual cell's exposure to growth media and the proper mixture of oxygen and carbon dioxide. Furthermore, suspension cultivation allows for a continuous cultivation of McCoy cells, as opposed to cultivation in batches.

The cells can be agitated and maintained in suspension by a variety of methods known in the art, including, for example, culture flasks, roller bottles, membrane cultures and spinner flasks. The cells may be kept in suspension during incubation by incubating the cells in a reactor employing a paddle, propeller or other means to agitate the culture and keep the cells contained therein in suspension. Using suspension cultures greatly facilitates keeping the cells actively growing and permits continuous culture expansion and scale-up.

In an embodiment, a second portion of the first culture is used for harvesting Lawsonia intracellularis bacteria. Thus, the first culture is not only used for passing pyroptotic McCoy cells to the second growth medium comprising fresh McCoy cells, but also for collecting Lawsonia bacteria for e.g. vaccine production. In this way, no separate cultures of McCoy cells for harvesting Lawsonia bacteria and for continuing growing Lawsonia bacteria are required. Instead, a substantially continuous method of culturing Lawsonia bacteria can be employed in which part of a culture of McCoy cells is used for continuing culturing of McCoy cells infected with Lawsonia bacteria, and another part of the culture is used for collecting Lawsonia bacteria.

Preferably, the second portion of the first culture is the total amount of first culture remaining after passing the first portion of first culture to the second growth medium. Harvesting can be done by techniques known in the art, such as centrifugation, filtration etc.

In an embodiment, the steps of determining a concentration of pyroptotic cells, passing a first portion of the first culture to fresh McCoy cells and growing the second culture are performed in a cycle. Here, the second culture of McCoy cells becomes the first culture of McCoy cells for a subsequent repetition or cycle of determining the concentration of pyroptotic McCoy cells, passing McCoy cells and growing a culture of McCoy cells. Or, in other words, in a first cycle, the second culture is a first “second culture”, and in a second repetition the second culture is a second “second culture” in the sense of the invention, etc.

Because for vaccine production large quantities of Lawsonia bacteria are required, one repetition of passing a first portion of the first culture McCoy cells to fresh medium containing fresh McCoy cells and growing a second culture of McCoy cells is generally not sufficient to provide enough Lawsonia bacteria to fulfill the demand for vaccine production. Thus, by performing multiple repetitions, the culturing of Lawsonia bacteria can be performed as a substantially continuous process and a higher yield can be obtained.

Furthermore, the first culture of McCoy cells is generally established from a frozen stock or seed of McCoy cells. Reviving these cells and scaling up to a volume suitable for growing McCoy cells and Lawsonia bacteria for vaccine production requires time and resources. Thus, by repeating the cycle of passing a first portion of the first culture McCoy cells to fresh medium containing fresh McCoy cells and growing a second culture of McCoy cells, a continuous process of growing Lawsonia bacteria, harvesting Lawsonia bacteria and inoculating a second culture with pyroptotic McCoy cells can be performed from a single initial starting culture.

Preferably the cycle is repeated 2-40 times, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 times.

The duration of each cycle may vary while repeating. Preferably, the cycle is repeated in a rhythm in days of 5-5-4, 5-4-5, 4-5-5, 4-3, or 3-4. In this way, the McCoy cells and Lawsonia bacteria can be grown in a continuous fashion while for example avoiding having to pass and/or harvest in weekends. For example, a rhythm of 5-5-4 means that the first “second culture” is grown for five days, the second “second culture” is grown for five days and the third “second culture” is grown for four days. This rhythm may then be repeated. It may also be followed by a different rhythm.

Preferably, the first portion of the first culture is in each cycle chosen such that the second culture of McCoy cells reaches a concentration of pyroptotic cells of 50-90% on a predetermined day, for example 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90%. In this way, the second culture of each cycle is suitable for passing pyroptotic cells to fresh McCoy cells for a subsequent cycle of passing and growing the McCoy cells.

In an embodiment, the first culture is grown for a period of 2, 3, 4, 5, 6, or 7 days. In this way, sufficient time is allowed for the McCoy cells and Lawsonia bacteria to grow before passing and/or harvesting.

In an embodiment, the second culture is grown for a period of 2, 3, 4, 5, 6, or 7 days. In this way, sufficient time is allowed for the McCoy cells and Lawsonia bacteria to grow before passing and/or harvesting.

In an embodiment, the concentration of fresh McCoy in the second growth medium is 1×10⁵-1×10⁷ cells/mL. Thus, sufficient cells are provided to develop into the second culture of McCoy cells infected with Lawsonia bacteria. Preferably, the fresh McCoy cells are provided in a volume of 50-2500 L of fresh medium, such as 100, 150, 200, 250, 300, 350, 400, 450, 500, 550, 600, 650, 700, 750, 800, 850, 900, 950, 1000, 1100, 1200, 1250, 1300, 1400, 1500, 1600, 1700, 1750, 1800, 1900, 2000, 2100, 2200, 2300, or 2400 L. 5×10⁵-5×10⁶ cells/mL, 6×10⁵-2.5×10⁶ or 7.5×10⁵-1×10⁶ fresh McCoy cells are for example provided in a volume of 2000 L of second growth medium.

In an embodiment, the step of growing the second culture of McCoy cells is followed by determining the concentration of pyroptotic McCoy cells in the second culture, passing a third portion of the second culture to third growth medium containing fresh McCoy cells to form a third culture of McCoy cells infected with Lawsonia intracellularis bacteria, such that the third medium is inoculated with 0.15-0.35 pyroptotic cells per fresh McCoy cell. The third culture of McCoy cells is grown at a dissolved oxygen concentration of less than 18%, and Lawsonia intracellularis bacteria are harvested from the third culture.

The third culture is for example the culture with which the method of growing Lawsonia bacteria is terminated, or a culture only intended harvesting Lawsonia bacteria but not for inoculating fresh McCoy cells. In such a case, it is no longer necessary to inoculate fresh McCoy cells with a first portion of the first culture of McCoy cells. Thus, the third growth medium containing fresh McCoy cells can be inoculated with a concentration of pyroptotic McCoy cells aiming for a maximal yield after growing the third culture. The volume of the third portion can for example be selected such that a ratio of pyroptotic McCoy cells:fresh McCoy cells in the third growth medium is 0.175:1, 0.2:1, 0.225:1, 0.25:1, 0.275:1, 0.3:1, or 0.325:1.

The first, second and third growth medium may have the same composition but may also be of a different composition, for example when the cultures are to be grown for a different number of days.

### Examples

### 1. Measuring the Yield of the Cultivation Process

### 1.1. Method

The yield of the cultivation process is determined using an enzyme-linked immunosorbent assay (ELISA), measuring antigenic mass being a polysaccharide associated with the outer cell membrane of Lawsonia intracellularis, as described in example 1 of EP2268308.

The wells of a polystyrene microtitre plate are coated with a monoclonal antibody (MoAb) against Lawsonia intracellularis. Serial three-fold dilutions of samples are incubated in the plates. Subsequently, the bound antigens are quantified using a second MoAb against Lawsonia intracellularis labelled with horse radish peroxidase and using 3,3′,5,5′-tetramethylbenzidine as substrate. The calibration line is drawn from the values of a reference standard with known antigenic mass which is included in each test. The antigenic mass of test samples in Units (U)/mL is determined against the antigenic mass of the reference standard.

### 1.2. Results

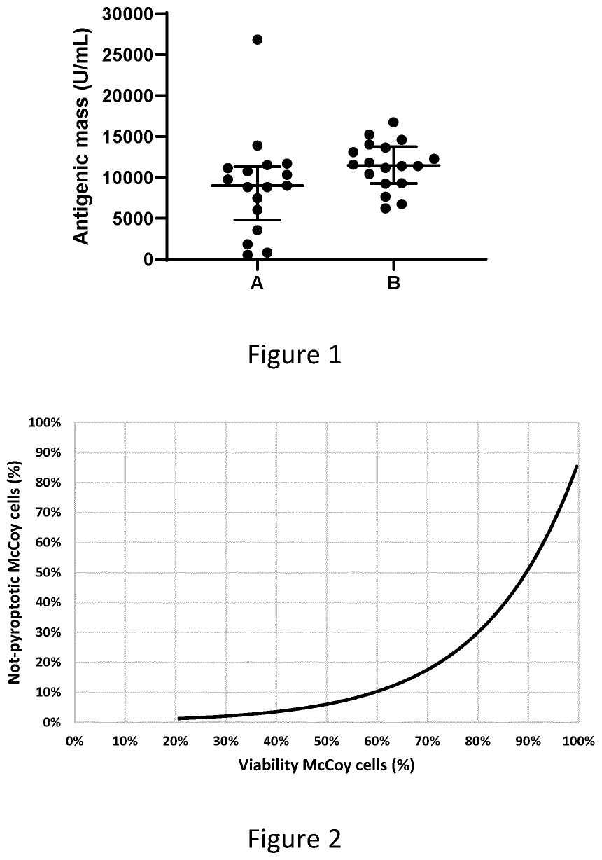

FIG. 1A shows the average yield from the second culture according to a method of culturing Lawsonia intracellularis bacteria according to the prior art, and the variability between repeats of the method according to the prior art. McCoy cells were grown in a 2000 L reactor. The concentration of Lawsonia bacteria present in the medium of the first culture was determined by means of dark field microscopy, and the first portion of the first culture was chosen such that the second growth medium containing 6×10⁵ fresh McCoy cells was inoculated with a multiplicity of infection (MOI) of 125. In other words, the second growth medium was inoculated with 125 Lawsonia intracellularis bacteria per fresh McCoy cell. The yield of the cultivation process was determined by ELISA after 5 days of growing.

FIG. 1B shows the average yield from the second culture according to a method of culturing Lawsonia intracellularis bacteria according to an embodiment of the invention, and the variability between repeats of the method according to the embodiment. McCoy cells were grown in a 2000 L reactor. The concentration pyroptotic McCoy cells in the first culture was determined, and the first portion of the first culture was chosen such that the second growth medium containing 6×10⁵ fresh McCoy cells was inoculated with 0.1 pyroptotic McCoy cell per fresh McCoy cell. The yield of the cultivation process was determined by ELISA after 5 days of growing.

As can be seen in FIG. 1, the method according to the invention results in a higher average yield in the second culture (about 40% higher), and in a more constant yield of the second culture as compared to the method according to the prior art.

### 2. Determining the First Portion Based on Caspase-1 Activation

### 2.1. Caspase-1 Activation Assay

Pyroptosis is triggered by caspase-1 activation. Pyroptotic cells are therefore characterized by active caspase-1. In order to measure caspase-1 activity and determine the concentration of pyroptotic McCoy cells in a culture, the FAM-FLICA™ (Bio-Rad, Hercules, USA) was used. Fluorescent labeled inhibitors of caspase-1 (FLICA) easily diffuse into the cell and bind covalently to active caspase-1.

According to the manufacturer's manual, 290 μL of 5×10⁵ cells/mL were plated in a 12-well plate. 10 μL 30× reconstituted FLICA solution was added to the wells and incubated in the dark for 30 minutes at 37° C., 5% CO₂ at 150 rpm. After incubation, cells were washed using 2 mL 1× apoptosis wash buffer, followed by centrifugation at 200 g for 5 minutes at room temperature. Cells were washed a second time with 1 mL 2× apoptosis wash buffer, followed by centrifugation at 200 g for 5 minutes at room temperature. The cell pellet was fixed using the fixative provided with the kit, and the cells were analyzed by flow cytometry using BD Facsverse™ (BD Biosciences, USA) and a 527/32 filter. 10,000 cells were acquired for each sample.

### 2.2. Selecting the First Portion Based on Caspase-1 Activation

The exact ratio of pyroptotic McCoy cells per fresh McCoy cells is determined depending on the volume of the vessel containing the fresh McCoy cells and the number of days the McCoy cells are to be grown in the second growth medium. The required volume of the first portion is then calculated based on the concentration of pyroptotic McCoy cells in the first culture and the exact ratio of pyroptotic McCoy cells per fresh McCoy cell with which the second growth medium is to be inoculated. In this way, the method can be adapted to culturing at different scales and to a desired length of a growth period, for example in order to avoid work on weekend days or to avoid depletion of medium.

### 3. Determining the First Portion Based on McCoy Cell Viability

### 3.1. Measuring Cell Viability

It was experimentally found that there was a correlation between the concentration of not-pyroptotic cells and the viability of the McCoy cells in the first culture, as shown in FIG. 2 for the strain of McCoy cells used.

Viability can be determined by directly measuring number of viable cells and the total number of cells. Cell viability can be measured e.g. using an assay measuring the protease activity of cells. CellTiter-Fluor™ Cell Viability Assay (Promega) makes use of a cell permeable fluorogenic protease substrate (GF-AFC) which is cleaved by proteases in live cells, resulting in a fluorescent signal proportional to the number of viable cells.

Viability can also be determined by measuring the number of not-viable cells and the total number of cells. In this case, a measure of viable cells can then be derived by subtracting the number of not-viable cells from the total number of cells. Not-viable cells can for example be stained using 4′,6-diamidino-2-phenylindole (DAPI) or propidium iodide (PI), and acridine orange can be used to stain the total of cells present in a sample. Commercially available systems, such as a Nucleocounter or ViCELL analyser can then be used to establish the viability of the first culture of infected McCoy cells. The fraction of not-viable cells consists of cells still in the process of dying, and cells that have already died. Thus, the number of viable cells (and the viability) may even be obtained by determining the total number of cells, the dead cells and the cells undergoing cell death. In this case, the number of viable cells is obtained by subtracting the number of dead cells and the number of cells undergoing cell death from the total number of cells.

### 3.2. Selecting the First Portion Based on McCoy Cell Viability

The concentration of not-pyroptotic cells can be determined quickly and conveniently by measuring the viability of the McCoy cells in the first culture and deriving the number of not-pyroptotic cells present in the first culture based on the total number of cells, the number of (not-) viable cells in the first culture, and the correlation shown in FIG. 2. The number of pyroptotic cells is then calculated by subtracting the number of not-pyroptotic cells from the total number of cells.

The exact ratio of pyroptotic McCoy cells per fresh McCoy cells is determined depending on the volume of the vessel containing the fresh McCoy cells and the number of days the McCoy cells are to be grown in the second growth medium. The required volume of the first portion is then calculated based on viability of the McCoy cells in the first culture and the exact ratio of pyroptotic McCoy cells per fresh McCoy cell with which the second growth medium is to be inoculated. In this way, the method can again be adapted to culturing at different scales and to a desired length of a growth period, for example in order to avoid work on weekend days or to avoid depletion of medium.

It was found that a first culture of McCoy cells having a viability of 40-60% was ideally suitable to inoculate growth medium containing fresh McCoy cells with pyroptotic McCoy cells from the first culture.

Table 1 gives the volume of first culture to be passed in case of a reactor comprising 2000 L of second growth medium containing 0.6×10⁶ fresh McCoy cells per mL, and wherein the second culture is to be grown for 5 days. Table 2 gives the volume of first culture to be passed in case of a reactor comprising 2000 L of second growth medium containing 0.6×10⁶ fresh McCoy cells per mL, and wherein the second culture is to be grown for 4 days. Table 3 gives the volume of first culture to be passed in case of a reactor comprising 50 L of second growth medium containing 0.6×10⁶ fresh McCoy cells per mL, and wherein the second culture is to be grown for 4-5 days.

TABLE 1
Volume of the portion of first culture to be passed
to second growth medium containing fresh McCoy cells
(in liters) in a reactor having a volume of 2000 L and
comprising 0.6 × 10⁶ fresh McCoy cells, when the
second culture is to be grown for 5 days.
2000 L - 5-day culture

| | Viable McCoy cell density (×10⁶ cells/mL) | | | | |
|---|---|---|---|---|---|
| | | 1.49-1.20 | 1.19-1.00 | 0.99-0.80 | 0.79-0.60 |
| VMcCoy cell | 100-95 | 380 | 400 | 400 | 400 |
| viability | 94-85 | 200 | 280 | 320 | 400 |
| (%) | 84-75 | 165 | 190 | 240 | 300 |
| | 74-65 | 150 | 160 | 200 | 260 |
| | 64-55 | 130 | 150 | 180 | 230 |
| | 54-45 | 120 | 140 | 170 | 220 |
| | <45 | 120 | 140 | 170 | 220 |

TABLE 2
Volume of the portion of first culture to be passed
to second growth medium containing fresh McCoy cells
(in liters) in a reactor having a volume of 2000 L and
comprising 0.6 × 10⁶ fresh McCoy cells, when the
second culture is to be grown for 4 days.
2000 L - 4-day culture

| | Viable McCoy cell density (×10⁶ cells/mL) | | | | |
|---|---|---|---|---|---|
| | | 1.49-1.20 | 1.19-1.00 | 0.99-0.80 | 0.79-0.60 |
| McCoy cell | 100-95 | 400 | 400 | 400 | 400 |
| viability | 94-85 | 300 | 400 | 400 | 400 |
| (%) | 84-75 | 250 | 285 | 360 | 400 |
| | 74-65 | 225 | 240 | 300 | 390 |
| | 64-55 | 195 | 225 | 270 | 345 |
| | 54-45 | 180 | 210 | 255 | 330 |
| | <45 | 180 | 210 | 255 | 330 |

TABLE 3
Volume of the portion of first culture to be passed
to second growth medium containing fresh McCoy cells
(in liters) in a reactor having a volume of 50 L and
comprising 0.6 × 10⁶ fresh McCoy cells, when the
second culture is to be grown 4-5 days.
50 L - 5-day and 4-day cultures

| | Viable McCoy cell density (×10⁶ cells/mL) | | | | |
|---|---|---|---|---|---|
| | | 1.49-1.20 | 1.19-1.00 | 0.99-0.80 | 0.79-0.60 |
| McCoy cell | 100-95 | 10 | 10 | 10 | 10 |
| viability | 94-85 | 10 | 10 | 10 | 10 |
| (%) | 84-75 | 8.3 | 9.5 | 10 | 10 |
| | 74-65 | 7.5 | 8 | 10 | 10 |
| | 64-55 | 6.5 | 7.5 | 9 | 10 |
| | 54-45 | 6 | 7 | 8.5 | 10 |
| | <45 | 6 | 7 | 8.5 | 10 |

As way of example, a sample of the first culture is stained with DAPI and acridine orange. The number of not-viable cells (positive for DAPI) and the total number of cells (positive for acridine orange) is determined using a Nucleocounter, and the viability and viable cell density of the McCoy cells of the first culture are calculated. The viability of the McCoy cells is calculated to be 80%, and the viable cell density of the McCoy cells is found to be 1.1×10⁶ cells/mL. Based on table 1, 190 L of the first culture is passed to a reactor containing 2000 L second growth medium comprising 0.6×10⁶ fresh McCoy cells/mL, and the resulting second culture of McCoy cells is grown for 5 days.

1. A method of culturing Lawsonia intracellularis bacteria, the method comprising:
a) infecting McCoy cells with Lawsonia intracellularis bacteria,
b) growing the infected McCoy cells in a first growth medium at a dissolved oxygen concentration less than 18% to arrive at a first culture of McCoy cells infected with Lawsonia intracellularis bacteria,
c) determining the concentration of pyroptotic McCoy cells in the first culture,
d) passing a first portion of the first culture to a second growth medium containing fresh McCoy cells, such that the second growth medium is inoculated with 0.05-0.25 pyroptotic McCoy cells per fresh McCoy cell, and
e) growing the McCoy cells in the second growth medium at a dissolved oxygen concentration less than 18% to arrive at a second culture of McCoy cells infected with Lawsonia intracellularis bacteria.

2. The method according to claim 1, wherein the second growth medium is inoculated with 0.075-0.2 pyroptotic cells per fresh McCoy cell.

3. The method according to claim 1, wherein a second portion of the first culture is used for harvesting Lawsonia intracellularis bacteria.

4. The method of claim 1, wherein the first portion of the first culture is chosen such that the second culture of McCoy cells reaches a concentration of pyroptotic cells of 50-90% on day 2-7 after passing the first portion of the first culture to the second growth medium.

5. The method of claim 1, wherein the McCoy cells are grown in suspension.

6. The method of claim 1, wherein the first culture and/or the second culture of McCoy cells is grown for a period of 2-7 days.

7. The method of claim 1, wherein the steps c) tot e) are performed 2-40 times.

8. The method according claim 7, wherein steps c) to e) are performed three consecutive times, and the second culture is two of three times grown for 5 days and one of three times grown for 4 days.

9. The method according to claim 7, wherein steps c) to e) are performed two consecutive times, and the second culture is grown for 3 days and 4 days, respectively.

10. The method of claim 1, wherein the concentration of fresh McCoy cells in step c) is 1×10⁵-1×10⁷ cells/mL.

11. The method of claim 1, wherein the concentration of fresh McCoy cells in step c) is 5×10⁵-7.5×10⁵ cells/mL.

12. The method of claim 1, wherein step e) is followed by
f) determining the concentration of pyroptotic McCoy cells in the second culture,
g) passing a third portion of the second culture to third growth medium containing fresh McCoy, such that the third growth medium is inoculated with 0.15-0.35 pyroptotic cells per fresh McCoy cell,
h) growing the McCoy cells in the third growth medium at a dissolved oxygen concentration less than 18% cells to arrive at a third culture of McCoy cells infected with Lawsonia intracellularis bacteria, and
i) harvesting the Lawsonia intracellularis bacteria from the third culture.

13. The method of claim 12, wherein the first and/or second and/or third culture of McCoy cells is grown at a dissolved oxygen concentration of 0-5%.

14. The method of claim 1, wherein the viability of the McCoy cells contained in the first portion of the first culture in step d) is 30-75%.
